(19) United States
(12) Patent Application Publication
(10) Pub. No.: US 2002/0150638 A1
(43) Pub. Date: Oct. 17, 2002

(54) Solid form compositions containing an extract of a raw material as the active ingredient and process of preparing the same

(76) Inventor: Nobuyoshi Kuboyama, Carlisle, MA (US)

Correspondence Address: Kevin S. Lemack, Nields & Lemack, 176 E. Main Street, Westboro, MA, 01581, US

(21) Appl. No.: 09/950,529

(22) Filed: Sep. 11, 2001

Related U.S. Application Data
(62) Division of application No. 09/777,737, filed on Feb. 5, 2001.

(51) Int. Cl. A61K 35/78

(52) U.S. Cl. 424/757; 424/725

(57) ABSTRACT

A process of preparing solid form of an extract of a raw material such as mung bean as the active ingredient, obtained by subjecting the raw material to extraction utilizing a heating, extracting and condensing system, and mixing the resulting liquid extract with a solution of a colloidal material such as agar-agar, and drying the same.

### BACKGROUND OF THE INVENTION

The present invention relates to solid form compositions and a process of preparing the same. The compositions are useful as cosmetics, perfumes, flavor enhancers, and as pharmaceuticals for treating and/or preventing a variety of diseases and conditions.

Apparatus disclosed in U.S. Pat. Nos. 5,572,923, 5,170,697 and 4,776,104, the disclosures of which are herein incorporated by reference, include extraction systems for extracting an effective ingredient from a material such as malt, soybean or the like. Such apparatus comprises a pulverizing minute particle generating tank including means for heating a reservoir of water to a predetermined temperature and a means for pulverizing or atomizing water; an extracting device connected to the pulverizing minute particle generating tank, which extracting device holds a raw material layer for adhering an effective ingredient of raw material to the pulverized minute particles as the pulverizing minute particles pass through the raw material layer; a condensing device connected to the extracting device for liquefying the pulverized minute particles that have passed through and extracted an effective ingredient from the raw material layer; a reserve tank into which the water liquefied at the condensing device empties; a blower provided in a path between the reserve tank and the pulverizing minute particle generating tank for decompressing the raw material layer within the extracting device; and a cooling means for cooling the condensing device and the reserve tank.

The resulting extract is in liquid form and heretofore has been impossible to solidify. Solidification would be advantageous, since pharmaceutical compositions are more easily prepared starting from solid forms rather than liquid. In addition, storage and shipment of the compositions is more cost efficient when in the solid form.

The present inventor has now found a process whereby the liquid extract prepared from the foregoing apparatus, and/or the extract prepared from apparatus having improved condensers and/or improved drying, can be easily solidified or dried. Pharmaceutical compositions, as well as other compositions useful in the preparation of cosmetics, perfumes and/or flavor enhancers, can be readily prepared from the solidified or dried extract.

### SUMMARY OF THE INVENTION

The problems of the prior art have been overcome by the present invention, which provides extracts in solid form, as well as compositions prepared from such extracts, possessing, inter alia, anti-cancer activity. The extracts of the present invention are obtained by utilizing a heating, extracting and condensing system that efficiently recovers the active ingredient(s) from a raw material. Preferably the condenser is made up of at least two preferably cylindrical containers, with at least one container having a cooling medium therein for condensing moisture from an air stream. An optional third container can be added. The resulting liquid extract is solidified using a colloidal material such as agar-agar. The process also allows for effective drying of the extract, to any degree desired. As a result, the moisture content of the extract can be carefully controlled. The process further allows for the preservation of effective ingredients in the raw material.

### BRIEF DESCRIPTION OF THE DRAWINGS

FIG. 1 is a schematic view of one embodiment of an extraction/drying apparatus used to extract the active ingredients) in accordance with the present invention;

FIG. 2 is a schematic view of another embodiment of the extraction/drying apparatus used to extract the active ingredients) in accordance with the present invention;

FIG. 3 is a fragmentary perspective view of an external cylinder of an extracting device used to extract the active ingredients) in accordance with the present invention;

FIGS. 4(a), (b) and (c) are perspective views showing the construction of the internal cylinder of an extracting device used to extract the active ingredient(s) in accordance with the present invention;

FIG. 5 is a plan view of air flow regulating means used in the extraction device used to extract the active ingredient(s) in accordance with the present invention;

FIG. 6 is a section view taken along lines XI-XI of FIG. 5;

FIG. 7 is a schematic view of an embodiment of a condensing device used in an extraction system used to extract the active ingredient(s) in accordance with the present invention; and

FIG. 8 is a schematic view of a condensing device used in an extraction system used to extract the active ingredient(s) in accordance with the present invention.

### DETAILED DESCRIPTION OF THE PREFERRED EMBODIMENTS

Suitable raw materials that can be subjected to the extraction system to produce the extract of the present invention include mung bean; soybean; coffee, including green coffee and roast coffee; lentil; green pea; pinto bean; black bean; adzuki bean; red kidney bean; navy bean; chick-pea; cannelini bean; ginseng (root); eucommia bark; mushroom (dried); malted barley; jalepeno pepper; mustard seed, sesame seed, celery seed, poppy seed, wild onion seed, paprika, cardamom, sugar and black pepper, and liquid raw materials such as juice from aloe, fruits, berries, caviar, and leaves and seeds. Mung bean (“phaseoulus aurcus”), soybean, coffee green and eucommia bark are particularly preferred, with mung bean being especially preferred.

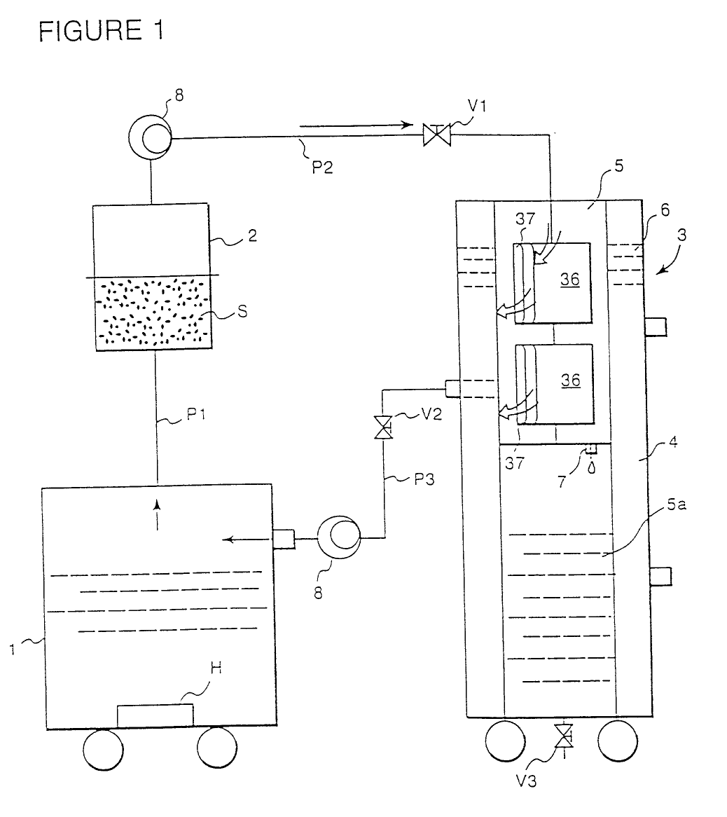

Hereinafter, a preferred method of obtaining the extract of the present invention will be described in more detail with reference to the accompanying drawings. FIG. 1 is a schematic view showing a construction of a first embodiment of the manufacturing apparatus, and in the drawing, reference numeral 1 is a housing or container having a reservoir of liquid, preferably water, therein. The housing 1 is preferably made of stainless steel. The size of the housing 1 is not particularly limited, and in the extraction embodiment shown, generally depends upon the amount of raw material 4 used and the desired rate of extraction of effective ingredient therefrom. The housing 1 includes means H for heating the reservoir, which means is not particularly limited, and can include an electric heating element or coil, a UV or IR heating element, a burner, etc. The heating means H must be sufficient to heat the liquid in the housing 1 to a temperature necessary to cause vaporization of the liquid. The heater can be coupled to a gauge (not shown) to allow the operator to specify the desired liquid temperature, and to a switch (not shown) to activate the heater. The heating means H can be located inside or outside of the housing 1. Means (not shown) can be optionally provided in association with the housing 1 to generate pulverized minute particles of water or a mist. Suitable means include an ultrasonic wave generating device comprising one or more sets (depending upon the tank size) of vibrators provided at the bottom of housing 1, each vibrator having the ability to pulverize water and create a mist. Conventional ultrasonic wave generators that are used in domestic ultrasonic humidifiers are suitable. Centrifugal atomization could also be used.

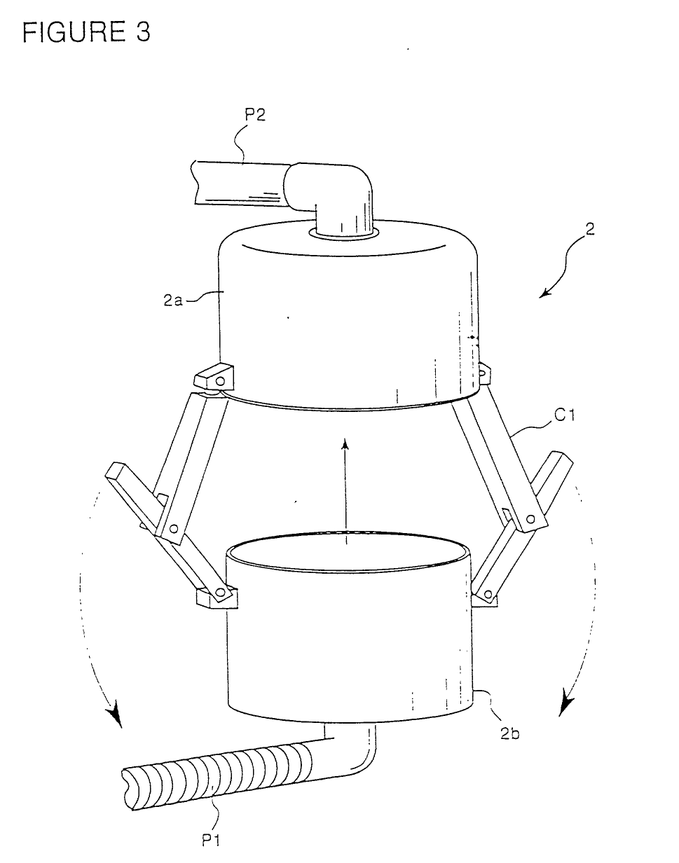

Housing 1 is in fluid communication via pipe P1 or the like with an extracting device 2 for extracting an effective ingredient from raw material S contained therein. FIG. 3 is a perspective view of the external appearance of the external cylinder which is the main element of the extracting device 2, and it includes a first external cylinder 2a and a second external cylinder 2b, both of which are constructed so as to be releasably joined to one another, and are preferably made of stainless steel. A temperature sensor (not shown) for detecting the temperature during the extraction operation can be fixed to the bottom side of the second external cylinder 2b. A hinged locking mechanism Cl joins cylinder 2a to cylinder 2b so that the raw material can be easily loaded and unloaded therefrom. FIG. 3 shows the extracting device 2 in its open, unlocked position.

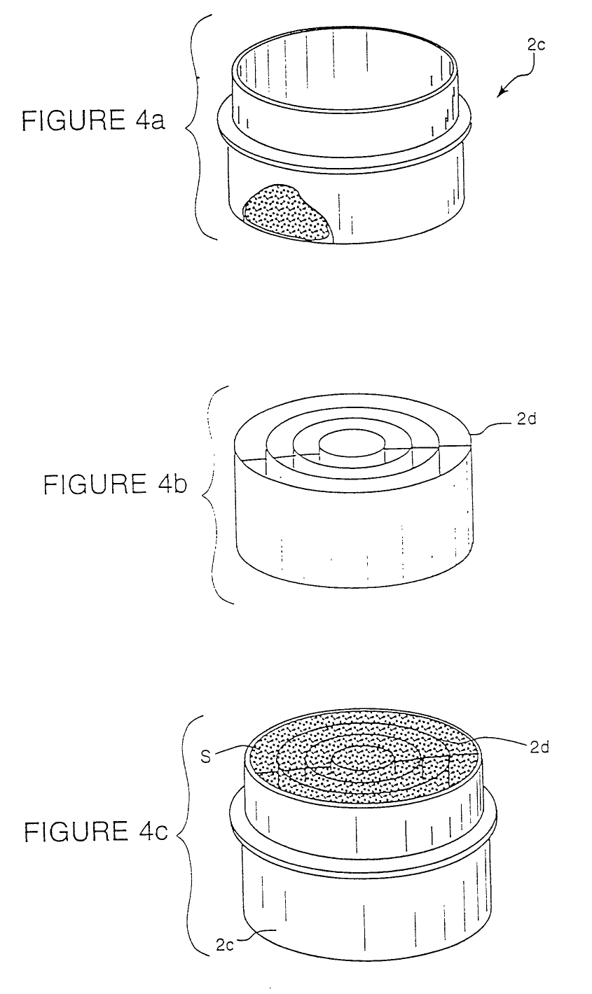

FIG. 4 is a schematic diagram of the internal cylinder that is housed in the external cylinder 2 of Figure 3. FIG. 4(a) shows internal cylinder 2c, which is of a suitable shape and size to fit into the aforementioned external cylinder 2, and includes at the bottom thereof a net portion for holding the raw material that has been crushed into small pieces. FIG. 4(b) shows a guide plate 2d for insertion into the internal cylinder 2c, and as shown in FIG. 4(c), it is constructed so as to partition the crushed pieces S of raw material such as mung beans, soybeans, malts or Korean ginseng in the interior of the internal cylinder 2c. The presence of this guide plate 2d allows the vaporized liquid from the housing 1 to easily and smoothly pass through the crushed pieces S of raw material as will be described below. Those skilled in the art will appreciate that other shapes for guide plate 2d may be used, such as a spiral shape.

The extracting device 2 is in fluid communication with condensing device 3 via pipe P2. A valve V1 can be positioned in pipe P2, and together with valve V2 in pipe P3 (discussed below), regulates the airflow to and the degree of decompression in condensing device 3. Preferably the condensing device 3 is comprised of two concentric cylinders; the outer cylinder 4 housing a cooling material to cool the contents of the inner cylinder 5. In the embodiment shown, the inner and outer cylinders are not co-extensive, thereby allowing for a lower inner portion 5a for collection of liquid condensate resulting from the cooling process. However, those skilled in the art will appreciate that the inner and outer cylinder 5 can be co-extensive, with suitable means (such as tubing in communication with the inner cylinder 5 at one end and with a supplementary container at the other) provided for condensate collection elsewhere. Similarly, the inner cylinder 5 could be smaller in length than the outer cylinder 4 in order to allow the cooling material contained in the outer cylinder 4 to surround not only the sides of the inner cylinder 5, but also the bottom thereof. In this latter embodiment, suitable means would again be provided for collecting the condensate elsewhere.

Preferably the cooling material 6 contained in the outer cylinder 4 is a liquid, such as water. However, the cooling material 6 can also be a gas or a solid such as ice or other material that can maintain a cold temperature for an extended period of time. The cooling material 6 can be circulated in the outer cylinder 4 to enhance cooling, and can be continuously or continually replenished during operation.

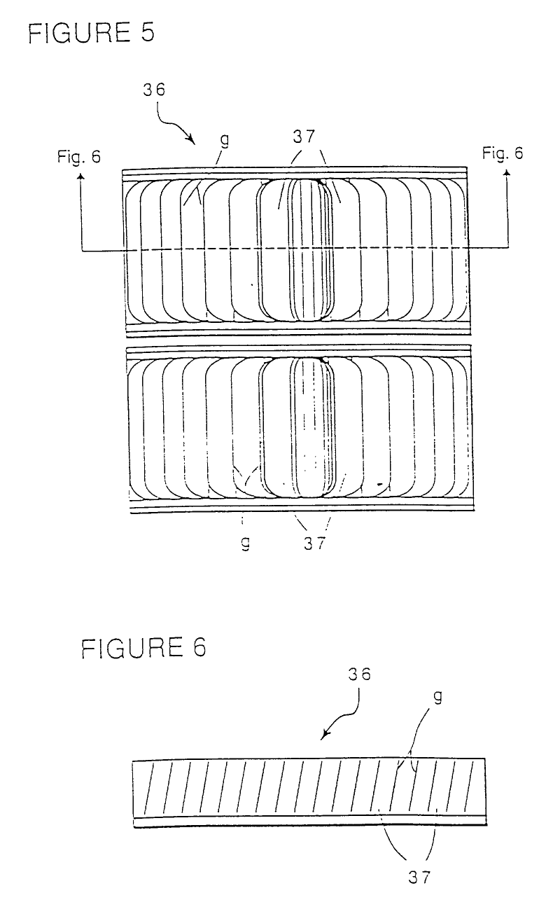

Preferably the inner cylinder 5 contains one or more airflow regulator means 36, most preferably two as shown. As illustrated in FIGS. 5 and 6, the air flow regulators 36 comprise a plurality of sloping plates 37 with a gap “g” formed between adjacent sloping plates 37. By adjusting the inclination of the sloping plates 37, it is possible to adjust the quantity of the airflow being regulated. Air flowing into the inner cylinder 5 causes the air flow regulators 36 to rotate about a vertical axis, thereby forcibly directing the air flow toward the wall of the cylinder 5 which is cooled by the cooling material 6 in the outer cylinder 4. Alternatively, the airflow regulator(s) 36 can be driven by a motor or the like to increase the extraction of the moisture from the air stream. Resulting condensate is drained from drain 7 and is collected.

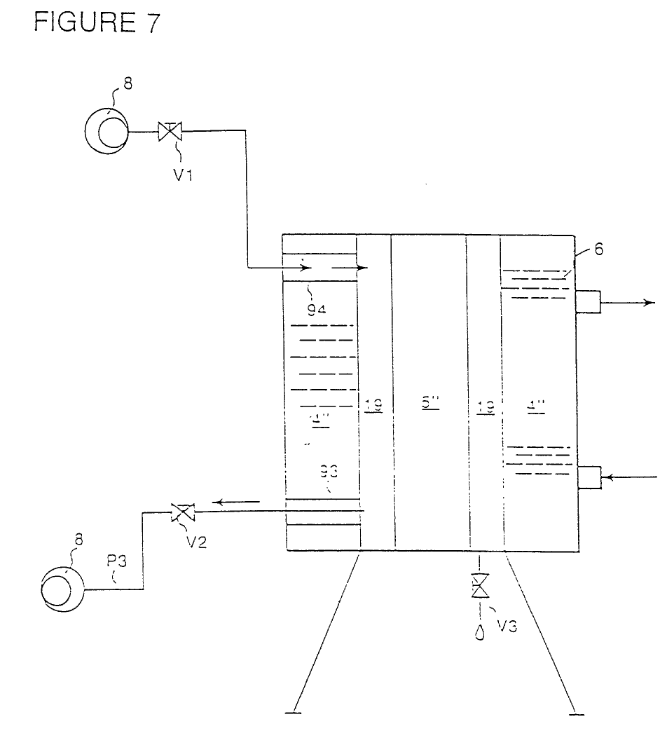

FIG. 7 illustrates an alternative embodiment of the condensing device 3 where airflow regulation is accomplished using a triple container design or the like. The outer container 4″ contains a cooling material 6 in its annulus, as in the previous embodiments. The middle container M receives the air flow from the extracting device via suitable piping 94, and the air flow proceeds out of the device (and optionally is recycled back to housing 1) via pipe 93. A central container 5″ is positioned so as to assist in directing the contents of the middle container M against the outer container 4″ to enhance cooling. The shape of the containers are preferably cylindrical but need not be; other shapes are suitable as long as cooling is enhanced such as by forcing the air in the middle container M against the outer container 4″. Surface area of the cooling walls is also important; thus a zig-zag shape could be used to increase surface area; or alternating projections could extend from the cooling walls to increase the surface area thereof.

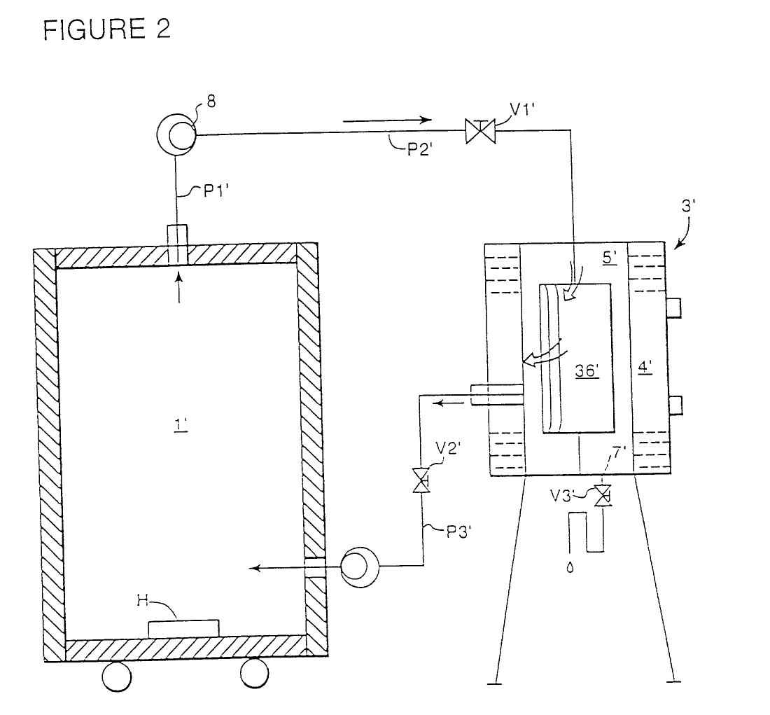

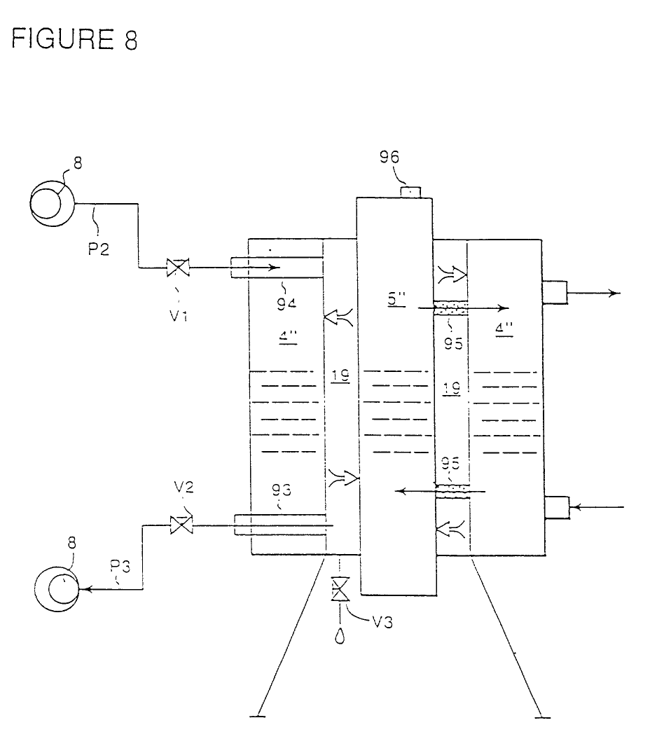

FIG. 8 illustrates a still further alternative embodiment of the condensing device. This embodiment is similar to that shown in FIG. 7, except the central container 5″ is filled with a cooling fluid, which can be the same or different from the cooling fluid contained in the outer container 4″. Where the fluid is the same, connecting means 95 can be provided between the central container 5″and the outer container 41″ to circulate the cooling fluid therebetween. As in the embodiment of FIG. 7, the central container 5″ is preferably but need not by cylindrical; other shapes that enhance cooling by increasing the surface area of the cooling surfaces and assist in forcing the medium to be cooled against the cooling surfaces can be used. The central container 5″ can also be made shorter so that the medium to be cooled is also exposed to the bottom of the container. In addition, the inlet and outlet for the medium to be cooled can be located so that the medium to be cooled travels around the perimeter of the central container 511 prior to its exit from the condensing device. As in from FIG. 9, the central container 5″ also can be longer than the outer container 4″ and middle container M, and includes an inlet 96 for introducing the cooling fluid therein. The condensing device can be combined with a heater to increase the temperature of the medium from which moisture is being removed. A plurality of the devices can be arranged in series to enhance condensing, and can be arranged in series either vertically or horizontally, depending in part on space considerations. The device is easier and faster to manufacture than the embodiment of FIG. 2 using the rotary device to regulate airflow.

At least one or more (two shown) air circulating or driving means is provided, preferably in the form of a fan or blower 8. The fan(s) 8 should be of a sufficient size so as to create decompression and provide flow through the system. The decompression should be within the range of about 5 to 500 mm H₂O. A conventional domestic vacuum cleaner fan has been found to be effective.

The condensing device 3 is in communication with housing 1 via pipe P3. Valve V2 can be positioned in pipe P3 to regulate airflow and decompression with valve V1. For example, if valve V1 is partially closed while valve V2 is open, then the condensation apparatus 3 will be under a state of decompression. If valve V2 is partially closed while valve V1 is open, the pressure in the condensation apparatus 3 will increase. The modulating of the valves can be accomplished manually or automatically.

The operation of the apparatus will now be described based upon the above construction.

First, the raw material is crushed to a magnitude approximating rice grains by any suitable means and is filled into the internal cylinder 2c illustrated in FIG. 5(a). Once filled, the net is placed over the raw material in order to stably maintain it in the internal cylinder 2c.

Successively, the internal cylinder 2c is inserted into the external cylinder 2 shown in FIG. 3. The housing 1 is filled with a sufficient amount of water or other liquid so that a mist can be produced. The water can be maintained at the same level continuously, or can be added batchwise. The temperature gauge is set to the desired temperature, and the heater is activated to heat the water to a suitable temperature such that the temperature in the extracting device 2 is at such a level (generally below 100° C.) as to not destroy the effective ingredients of the raw material. For example, in the case of mung beans and soybeans, the temperature of the water is preferably heated to about 85° C., so that the temperature of the water when it reaches the extracting device is between about 60-70° C., preferably about 65° C.

Once the water temperature in the housing 1 reaches the desirable level, the blower(s) 8 is activated to initiate flow through the system. The blower(s) 8 causes air flow to circulate in the closed circulating path formed by the housing 1, the extracting device 2 and the condensing device 3, as well as the pipes connecting these respective devices. The mist of water generated in the housing 1 thus pass through pipe P together with the airflow and reaches the extracting device 2. The temperature in the extracting device 2 can be measured by a temperature sensor to ensure that the appropriate temperature is reached therein. The temperature in the housing 1 can be controlled in response to the temperature in the extraction device 2.

As described above, the airflow is circulated between each device by the operation of the blower(s) 8, but since the extracting device 2 is filled with the crushed particles of raw material, the raw material creates a resistance to the air flow, thereby creating a decompressed space within the extracting device 2.

Once the decompressed state is achieved, ingredients within the raw material are extracted to the surface of the crushed pieces S of raw material, and are then captured by the mist of water passing therethrough. Since the temperature within the extracting device, and more particularly, the temperature within the internal cylinder 2c is maintained within the desired range, the ingredients contained in the raw material are extracted into the water without being destroyed by heat.

The resulting liquid (e.g., water) containing the effective ingredient of the raw material then flow to the condensing device 3 through the connecting pipe P2 together with the air flow from the blower 8. The outer cylinder 4 of the condensing device 3 is filled with cooling material, preferably water, at a temperature sufficient to cause condensation of the water in the inner cylinder 5. Airflow and decompression in condensing device 3 are controlled by modulation of valves V1 and V2. The liquefied or condensed material drains through drain 7 as shown, and can be ultimately collected through valve V3.

The particles that are not liquefied in the condensing device 3 are sucked towards the housing 1 through the connecting pipe P3 together with the airflow, and are thereby recycled. The recycled portion optionally can be preheated such as by a rectifying plate or spiral shape, so as not to lower the temperature of the water in the tank 1.

The cooling material in the condensing device 3 can be changed periodically. Alternatively, a continuous flow of cooling liquid can be used to cool the inner cylinder 5.

The raw material can be crushed to about the size of rice grain. However, the concentration of effective ingredient contained in the final product can be controlled by varying the size of the raw material. For example, if the raw material is crushed into fine pieces, a final product high in effective ingredient concentration can be obtained. However, in such a case the rate at which the final product is produced decreases. As the size of the raw material increases, the concentration of effective ingredient in the final product decreases, and the rate of production increases. Similarly, the use of the guide plate 2d increases the yield of final product per hour by about 20%, but the concentration of effective ingredient in the final product decreases.

With the foregoing apparatus described in each of the embodiments, it is possible to obtain balanced drying without influence from external air by circulating moisture-laden air through a condensing device to reduce or eliminate the moisture content thereof. The result is a substantial reduction in drying time and concomitant energy requirements therefor.

The product is a colorless, transparent and clear liquid. In the case of mung bean, for example, the composition of the extract is as follows (a suitable range is also listed, since the precise concentration of ingredients may vary slightly depending on the source of the raw material):

| | Concentration | |
|---|---|---|
| Compound | (ppm) | |
| acetone | 0.051 | 0.02-0.08 |
| isobutanol | 0.451 | 0.15-0.75 |
| butanol | 0.775 | 0.47-1.07 |
| 2-methylbutanol | 1.483 | 1.18-1.78 |
| 3-methylbutanol | 2.122 | 1.82-2.42 |
| pentanol | 2.163 | 1.85-2.46 |
| acetoin | 0.272 | 0.01-0.57 |
| 2,6-dimethylpyrazine | 0.576 | 0.27-0.87 |
| hexanol | 8.309 | 5.3-11.3 |
| 2-hydroxy-2-methyl-4-pentanone | 0.349 | 0.04-0.64 |
| ethylene glycol monbutyl ether | 2.303 | 2.0-2.6 |
| N,N-dimethylacetoamide | 0.498 | 0.19-0.79 |
| 2-ethylhexanol | 2.828 | 2.50-3.12 |
| 2-(methylthio)ethanol | 1.534 | 1.23-1.83 |
| isophorone | 1.296 | 0.99-1.59 |
| 2-hydroxy-2,6,6-trimethylcyclohexanone | 0.411 | 0.11-0.71 |
| γ-valerolactone | 0.924 | 0.52-1.22 |
| γ-butyrolactone | 3.677 | 3.27-4.07 |
| diethylene glycol monoethyl ether | 1.367 | 1.15-1.56 |
| 3-furfuryl alcohol | 0.771 | 0.47-1.07 |
| γ-hexalactone | 1.813 | 1.52-2.11 |
| 2-phenyl-2-propanol | 0.960 | 0.66-1.26 |
| diethylene glycol monobutyl ether | 0.386 | 0.05-0.68 |
| styralil alcohol | 0.560 | 0.26-0.86 |
| benzyl alcohol | 25.976 | 21.9-29.9 |
| phenylethyl alcohol | 12.9696 | 9.9-15.9 |
| maltol | 2.741 | 2.44-3.04 |
| phenol | 1.550 | 1.25-1.85 |
| methyleuganol | 0.708 | 0.4-1.0 |
| γ-nonalactone | 0.295 | 0.01-0.59 |
| pantolactone | 0.308 | 0.01-0.5 |
| β-phenoxyethanol | 0.933 | 0.53-1.23 |
| eugenol | 1.593 | 1.29-1.89 |
| nonanoic acid | 0.783 | 0.48-1.08 |
| 3-ethyl-4-methyl-1H-pyrrole-2,5-dione | 0.353 | 0.05-0.65 |
| 2-amino-benzonitrile | 0.209 | 0.01-0.5 |
| dedecanoic acid | trace | trace |

The extract is then solidified or dried according to the process of the present invention. The procedure for solidification or drying is described below.

A colloidal material is chosen as the solidification or drying vehicle. Suitable colloidal materials include agar-agar, gelatin made from an animal source, cornstarch or flour, or other materials that upon dissolving and drying, will form a film. The preferred colloidal material is agar-agar, which is a vegetable gel of select edible seaweed (gelidium), commercially available from Shirakiku Eden Foods or Nishmoto Trading Co., and this material will be used as the example hereinafter.

The agar-agar is washed with potable water (other colloidal materials, such as cornstarch or gelatin, do not require washing or subsequent soaking and removal of excess water). More specifically, one bar of agar-agar (or about 7 grams) is washed with water repeatedly and by changing the water several times, preferably three times. Debris or other contaminants that may be embedded in the agar-agar are thus removed. The agar-agar optionally can be bleached using a suitable bleaching agent, if desired. If bleach is used, the agar must be rinsed well after bleaching in order to ensure that the bleaching agent is removed.

Once washed, the agar-agar is soaked in potable water for about 10 minutes to about one hour. Ten to fifteen minutes is the time necessary to soften the agar-agar. Additional soaking is preferred, in order to minimize any effect that the ingredients in the agar-agar might have on the final product. The water is then drained, and any excess moisture is removed by squeezing the agar-agar or by other suitable means (such as by spinning the agar-agar in a conventional salad spinner device or the like).

The resulting agar-agar is torn into small pieces and allowed to dry, such as by increasing its surface area by spreading it out on a large screen through which moisture can drip through into a receptacle. The drying period may last from about 10 to about 15 minutes, and may be carried out at room temperature.

Separately, a liquid vehicle, preferably water (such as tap water, filtered tap water, distilled water, and/or bottled water) is brought to a boil. Once boiling, the dried agar-agar is added while stirring. In the case where about 7 grams of agar-agar are used, about 8 ounces of water is suitable for this purpose. Stirring is continued until the agar-agar is completely dissolved in the liquid vehicle (generally about 3-5 minutes).

The dissolved agar is transferred to another receptacle, and about one pint (473 ml) of the liquid extract (preferably at room temperature) is added into the same receptacle while vigorously stirring to ensure that the liquid extract mixes uniformly with the agar solution. It is preferred that boiling of the extract be avoided, in order to preserve its effective ingredients(s).

The resulting mixture is cooled and then allowed to dry in a suitable vessel or on a suitable surface at or about room temperature. Preferably a shallow vessel is used, so that the mixture is spread over a larger surface to enhance drying efficiency. Those skilled in the art will appreciate that the depth of the vessel determines, in part, the thickness of the resulting dried solid. During this drying process, when the mixture cools to a lukewarm temperature, the vessel can be transferred to a refrigerator or the like to solidify in a cooler than room temperature environment. The drying process can be stopped at any time if some predetermined amount of moisture is desired in the final product.

Once the mixture solidifies (about 30 to 60 minutes after refrigeration) or drying is taken to its desired extent, the solid can be removed from refrigeration and spread onto a screen at room temperature. If desired, the solid can be broken up into smaller pieces such as by cutting in order to enhance room-temperature (60-65° F.) drying. If desired, a fan or the like can be used to blow air onto the solid to enhance drying. Preferably the drying is completed at a temperature no higher than room temperature, so as to avoid deleteriously effecting the active ingredient(s). Drying is usually complete within about 1 to 2 days. Again, the drying can be terminated sooner if some amount of moisture is desired in the final product. Where increased heat is not deleterious to the final product, a dryer at about 50° C. or less can be used.

The resulting dried product is a paper-thin solid sheet of material. This can be ground using a conventional grinder to produce a solid powder. If desired, prior to grinding, the solid sheet can be placed in a dryer at 60-70° C. for a few minutes to ensure that all moisture is removed. This may facilitate the grinding operation and may also make the resultant powder finer.

The yield of powder is about 5-7 grams. It is a condensed and/or concentrated form of the liquid extract. Transportation and storage are facilitated and made more cost effective. The effective ingredients in the extract are preserved. The powder also has a longer shelf life than the liquid extract, and lends itself to chemical identification and testing.

The present invention also can be utilized in cases where the starting material to be extracted is not suitable for the extraction system described. For example, the ideal raw material is granular or particulate, such as the size of rice grains, to provide effective surface area for extraction. However, certain raw materials (such as soil, sesame seeds, celery seeds, poppy seeds, wild onion seeds, paprika and cardamom) do not meet these criteria, as they may be composed of particles that are too small, for example, for effective extraction. Accordingly, such raw materials can first be subjected to the solidification treatment with colloidal material as described above, and then subject to the extraction treatment once granulized, and then optionally solidified again.

A further application of the solidification treatment of the present invention is with liquid raw materials such as blood, aloe (juice), fruits (juice), berries (juice), caviar, juice from different leaves and seeds, etc. The liquid raw materials can be solidified according to the process of the present invention, and then the resulting solid can be subjected to the extraction treatment, and then optionally solidified again.

The pharmaceutical compositions of the present invention are useful as human and animal drugs, such as for the treatment and/or prevention of various diseases and conditions, including cancer, reducing metastasis and neoplastic growth, leukemia, kidney disease, liver disease, including hepatitis, diabetes, atopic dermatitis, high blood pressure, high cholesterol, arthritis, rheumatoid arthritis, AIDS, head injuries, Alzheimer's disease, ear discharge, Lyme disease, etc.

The magnitude of the therapeutic or prophylactic dose of the extracts of the present invention in the treatment or prevention of disease will depend in part upon the identity, severity and nature of the condition being treated. The dose and the frequency of the dosing will also vary according to age, body weight and response of the particular patient. In general, the total daily dose range for the active ingredient(s) of the present invention is 5-10 ml two to three times a day. The dose for more severe conditions can be 30-60 ml., three to four times daily. Initial dosage for severe conditions can be as high as about 240 mls., three to four times daily for a week to ten days, and then reduced to 30-60 mls. three to four times a day. Moderate dosages can be about 120 mls., twice daily.

Any suitable route of administration well known to those skilled in the art may be employed to provide an effective dosage of the active ingredient(s) of the present invention, although oral administration is preferred, most preferably in liquid form.

The pharmaceutical compositions of the present invention may be combined with other therapeutic agents, such as analgesics.

The pharmaceutical compositions of the present invention are administered to animals, including dogs, cats, fish and humans. The compounds of the present invention can include pharmaceutically acceptable carriers and other conventional additives, including aqueous based carriers, co-solvents such as ethyl alcohol, propylene glycol and glycerin, fillers, lubricants, wetting agents, flavoring agents, coloring agents, emulsifying, suspending or dispersing agents, suspending agents, sweeteners, etc. Preferably the extract is simply diluted with water and administered orally without any carriers or additives.

The extract refined from the raw material has a noticeable efficacy.

### EXAMPLE

U.S. grown organic Mung Bean was ground to the size of approximately rice grains. Three pounds of the ground Mung Bean were placed in the inner container of the extraction system shown in FIG. 4. Water in the reservoir was heated to 850° C., and the blower was started to circulate the air. The temperature of the inner container reached approximately 65° C. The effective ingredient(s) from Mung Bean was extracted, cooled and collected in a container. After approximately thirty minutes, the blower was stopped and the collected liquid (approximately 1.2 liters) was obtained. It was a colorless, transparent and clear liquid. It was filtered to remove any sediment.

The resulting extract can be orally administered to an animal in an effective amount with or without further dilution.

What is claimed is:

1. A method for preparing a solid form of an extract of a raw material, comprising extracting from said raw material an extract by the following method:
heating water to a predetermined temperature;
atomizing said heated water into minute particles;
contacting said raw material under a state of decompression with said heated and atomized water particles;
condensing the resulting water particles; and
collecting the resulting cooled water; and solidifying the resulting liquid extract by the following method:
providing a predetermined quantity of a colloidal material;
adding said colloidal material to boiling water;
adding to said boiling water and colloidal material mix said extract while stirring;
drying the resultant mix to produce a solid form of said extract.

2. The method of claim 1, wherein said colloidal material is agar-agar.

3. The method of claim 2,

4. The method of claim 1, further comprising grinding said solid form in to a powder.

5. The method of claim 1, wherein said raw material comprises mung bean.

6. The method of claim 1, wherein said raw material comprises soy bean.

7. The method of claim 1, wherein said raw material comprises coffee.

8. The method of claim 1, wherein said raw material comprises eucommia bark.

9. The solid extract obtained by the method of claim 1.

10. The solid extract obtained by the method of claim 2.

11. A method of increasing the surface area of a raw material, comprising:
providing a predetermined quantity of a colloidal material;
adding said colloidal material to boiling water;
adding to said boiling water and colloidal material mix said raw material while stirring; and
drying the resultant mix to produce a solid form of said raw material.

12. The method of claim 11, wherein said colloidal material is agar-agar.

13. The method of claim 12, further comprising the steps of: washing said colloidal material with water;
soaking said washed colloidal material in water to soften said colloidal material; and
removing any excess moisture from said softened colloidal material; prior to adding said colloidal material to said boiling water.

14. A method of solidifying a liquid raw material, comprising:
providing a predetermined quantity of a colloidal material;
adding said colloidal material to boiling water;
adding to said boiling water and colloidal material mix said raw material while stirring; and
drying the resultant mix to produce a solid form of said raw material.

15. The method of claim 14, wherein said colloidal material is agar-agar.

16. The method of claim 15, further comprising the steps of: washing said colloidal material with water;
soaking said washed colloidal material in water to soften said colloidal material; and removing any excess moisture from said softened colloidal material; prior to adding said colloidal material to said boiling water.

17. The method of claim 14, wherein said resulting solid form of said raw material is further subjected to an extraction process, said extraction process comprising:
heating water to a predetermined temperature;
atomizing said heated water into minute particles;
contacting said raw material under a state of decompression with said heated and atomized water particles;
condensing the resulting water particles; and collecting the resulting cooled water.
